(19) United States
(12) Patent Application Publication
(10) Pub. No.: US 2006/0264344 A1
(43) Pub. Date: Nov. 23, 2006

(54) Mild foaming cleansing composition

(75) Inventors: Jessica Weiss Goldberg, Fairfield, CT (US); Shauna Mary Lagatol, New Haven, CT (US); Ewa Urszula Padlo, Norwalk, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division Of Conopco, Inc.

(21) Appl. No.: 11/135,243

(22) Filed: May 23, 2005

(51) Int. Cl. A61K 8/00

(52) U.S. Cl. 510/130

(57) ABSTRACT

A foaming mild liquid cleansing composition that is dispensed as a mousse or foam is described that contains very low levels of surfactant and that can be conveniently wiped off the skin while depositing significant amounts of skin conditioning compounds. In a preferred embodiment, the composition is provided as a transparent microemulsion.

### BACKGROUND OF THE INVENTION

1. Field of the Invention

The present invention relates to detergent compositions suitable for topical application for cleansing the human body, such as the skin and hair. In particular, it relates to a mild foaming wipe-off composition.

2. The Related Art

U.S. Pat. No. 5,494,533 issued to Woodin Jr. et al. on Feb. 27, 1996 discloses an aqueous cleansing system including a propellant-free mechanical foaming device. U.S. Pat. No. 6,730,288 issued to Abram on May 4, 2004 discloses an aerosol foaming cleanser containing high levels of petrolatum which is an occlusive lipophilic (hydrophobic) emollients. U.S. Pat. No. 6,407,044 issued to Dixon, on Jun. 18, 2002 discloses aerosol personal cleansing compositions containing appreciable levels of anionic surfactant and and lipophilic skin moisturizing agents.

PCT publication no. WO9938491 to Dixon, et al. published Aug. 5, 1999 discloses moisturizing personal cleansing compositions with substantial levels of anionic surfactants and lipophilic emollients. US Publication no. 2002/028182A1 (PCT publication no. WO9827936) to Dawson, et al., published Mar. 7, 2002 discloses an aerosol cleansing composition with at least 18% by wt. of an anionic surfactant. U.S. Pat. No. 5,002,680 to Schmidt et al. issued Mar. 26, 1991 discloses a mild skin cleansing aerosol mousse with substantial levels of surfactants including anionic surfactants.

Surprisingly, a mild, foaming aerosol cleansing composition with low surfactant content has been found to cleanse and moisturize the skin and hair without the need for appreciable levels of surfactants, especially harsh anionic surfactants, and/or substantial amounts of lipophillic emollients that are disclosed in the prior art. The inventive composition may be advantageously wiped off without the need to be washed off and is therefore convenient for travel. Furthermore the inventive cleansing composition surprisingly forms dense foam on dispensing from either an aerosol dispenser or a mechanical foamer dispenser.

### SUMMARY OF THE INVENTION

In one aspect the present invention is a a mild foaming cleansing composition including but not limited to:
a. amphoteric surfactant(s) in a total concentration range of about 0.1 to 5% by wt.;
b. optionally anionic surfactant(s) in a total concentration that is less than the amphoteric surfactant(s);
c. propellant(s) in a total concentration range of about 0 to 15% by wt., and
d. less than about 5% by wt. of a hydrophobic skin conditioning agent.

In a further aspect of the invention is a mild mousse foaming packaged cleansing product including but not limited to a container having a valve, a low surfactant cleansing composition contained in the container including: amphoteric surfactant(s) in a total concentration range of about 0.1 to 5% by wt.; optionally anionic surfactant(s) in a total concentration that is less than the amphoteric surfactant(s); propellant(s) in a total concentration range of about 0.5 to 10% by wt., and less than about 5% by wt. of a hydrophobic skin conditioning agent, and wherein the valve is capable of releasing the cleansing composition when the internal pressure exerted by the cleansing composition is greater than the outside air pressure.

In another aspect of the invention is a mild foaming packaged cleansing product including but not limited to a container having a non-aerosol mechanical dispenser, a low surfactant cleansing composition contained in the container including: amphoteric surfactant(s) in a total concentration range of about 0.1 to 5% by wt.; optionally anionic surfactant(s) in a total concentration that is less than the amphoteric surfactant(s); and less than about 5% by wt. of a hydrophobic skin conditioning agent, and wherein the dispenser is capable of dispensing the cleansing composition as a foam

In yet another aspect of the invention is a method for cleansing the skin without rinsing with water, including but not limited to the steps of: applying the mild foaming cleansing composition of claim 1 to the skin; distributing the applied composition to the area of skin desired; and wiping off the applied composition.

### DETAILED DESCRIPTION OF THE INVENTION

In one aspect the present invention is a a mild foaming cleansing composition including but not limited to:
a. amphoteric surfactant(s) in a total concentration range of about 0.1 to 5% by wt.; (preferably at a maximum concentration of about 4, 3, 2 or 1% by wt.)
b. optionally anionic surfactant(s) in a total concentration that is less than the amphoteric surfactant(s); (preferably less than about 0.5, 0.4, 0.3, or 0.1% by wt. and advantageously greater than about 0.01 or 0.05% by wt.)
c. propellant(s) in a total concentration range of 0 to about 15% by wt., and
d. less than about 5% by wt. of a hydrophobic skin conditioning agent.

Preferably the propellant is present in the inventive composition at a minimum concentration of about 0.5, 1, 2 or 3% by wt. and at a maximum concentration of about 5, 6, 7, 8, 9 or 10% by wt. for an aerosol system. Propellants are substantially absent in the case where a mechanical foamer device is used.

Advantageously, the inventive composition further includes hydrophilic emollients in the concentration range of about 0.1 to 10% by wt. Preferably the hydrophilic emollients include at least about 1% by wt. of C3 to C6 diol(s) or triol(s) or blends thereof such as glycerin, propylene glycol and the like.

Preferably the propellants are selected from hydrocarbons (including n-butane, isobutane, n-propane, isopropane, n-pentane, isopentane and mixtures thereof), dimethyl ether, carbon dioxide, nitrous oxide, compressed air, nitrogen, fluorohydrocarbons, or blends thereof and the like.

In a preferred embodiment, the composition is a microemulsion. Advantageously the total surfactant concentration of the inventive composition is less than about 5% by wt. Preferably the nonionic surfactant concentration is in the range of 0 to about 5% by wt., more preferably at a minimum concentration of about 0.1, 0.2, 0.3 or 0.5%, by wt to maximum concentration of about 2, 3, or 4% by wt.

Advantageously the density of the foam dispensed by a dispenser measured 5 seconds after dispensing is at least about 0.1 g/ml (at approx. 23 C and approx. 760 torr or 101 Kpa), preferably at least about 0.2 g/ml (at the same conditions).

In a preferred embodiment, the soap content of the inventive composition is less than about 5% by wt, more preferably less than about 4, 3, 2, 1 or 0.5% by wt. Advantageously inventive composition further includes one or more skin active agent(s) such as antibacterial agent(s), anti-aging agent(s), anti-acne agent(s) or skin firming agent(s) or blends thereof and the like in the total concentration range of about 0.02 to 3% by wt.

In a further aspect of the invention is a mild mousse foaming packaged cleansing product including but not limited to a container having an valve, a low surfactant cleansing composition contained in the container including: amphoteric surfactant(s) in a total concentration range of about 0.1 to 5% by wt.; optionally anionic surfactant(s) in a total concentration that is less than the amphoteric surfactant(s); propellant(s) in a total concentration range of about 0.5 to 10% by wt., and less than about 5% by wt. of a hydrophobic skin conditioning agent, and wherein the valve is capable of releasing the cleansing composition when the internal pressure exerted by the cleansing composition is greater than the outside air pressure.

In another aspect of the invention is a mild foaming packaged cleansing product including but not limited to a container having a non-aerosol mechanical dispenser, a low surfactant cleansing composition contained in the container including: amphoteric surfactant(s) in a total concentration range of about 0.1 to 5% by wt.; optionally anionic surfactant(s) in a total concentration that is less than the amphoteric surfactant(s); and less than about 5% by wt. of a hydrophobic skin conditioning agent, and wherein the dispenser is capable of dispensing the cleansing composition as a foam

In yet another aspect of the invention is a method for cleansing the skin without rinsing with water, including but not limited to the steps of: applying the mild foaming cleansing composition of claim 1 to the skin; distributing the applied composition to the area of skin desired; and wiping off the applied composition.

Surfactants:

Surfactants are an essential component of the inventive foaming cleansing composition. They are compounds that have hydrophobic and hydrophilic portions that act to reduce the surface tension of the aqueous solutions they are dissolved in. Useful surfactants can include anionic, nonionic, amphoteric, and cationic surfactants, and blends thereof. Total surfactant(s) concentration will preferably range from about 0.5 to about 5% by weight of the foaming cleansing composition. Preferably, this component is present from about 1% to about 2% in the foaming cleansing composition.

Amphoteric Surfactants

One or more amphoteric surfactants are used in this invention. They may be preferably used at concentrations of about 0.1, 0.2, 0.3 or 0.5, to about 1, 1.5, 2, 3, 4, or 5% by wt.

Such surfactants include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula:
R¹—[—C(O)—NH(CH₂)ₙ—]ₘ—N⁺—(R²)(R³)X—Y

where R¹ is alkyl or alkenyl of 7 to 18 carbon atoms;

R² and R³ are each independently alkyl, hydroxyalkyl or carboxyalkyl of 1 to 3 carbon atoms;

n is 2 to 4;

m is 0 to 1;

X is alkylene of 1 to 3 carbon atoms optionally substituted with hydroxyl, and
Y is —CO₂—or —SO₃—

Suitable amphoteric surfactants within the above general formula include simple betaines of formula:
R¹—N⁺—(R²)(R³)CH₂CO₂⁻

and amido betaines of formula:
R¹—CONH(CH₂)ₙ—N⁺—(R²)(R³)CH₂CO₂⁻

where n is 2 or 3.

In both formulae R¹, R² and R³ are as defined previously. R¹ may in particular be a mixture of C₁₂ and C₁₄ alkyl groups derived from coconut oil so that at least half, preferably at least three quarters of the groups R¹ have 10 to 14 carbon atoms. R² and R³ are preferably methyl.

A further possibility is that the amphoteric detergent is a sulphobetaine of formula:
R¹—N⁺—(R²)(R³)(CH₂)₃SO₃⁻
Or
R¹—CONH(CH₂)ₘ—N⁺—(R²)(R³)(CH₂)₃SO₃⁻

where m is 2 or 3, or variants of these in which —(CH₂)₃ SO₃⁻ is replaced by
—CH₂C(OH)(H)CH₂SO₃⁻

In these formulae R¹, R² and R³ are as discussed previously.

Amphoacetates and diamphoacetates are also intended to be covered in possible zwitterionic and/or amphoteric compounds which may be used such as e.g., sodium lauroamphoacetate, sodium cocoamphoacetate, and blends thereof, and the like.

Anionic Surfactants:

The foaming cleansing composition of the present invention may contain one or more anionic detergents. Preferably the anionic surfactant will be present at a lower concentration than the amphoteric surfactant. When used, the anionic surfactant may be present at about 0.1, 0.2, 0.3, 0.5, 0.7 or 1% by wt. to about 2, 3, 4, or 5% by wt.

The anionic detergent active which may be used may be aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate.

The anionic may also be an alkyl sulfate (e.g., C₁₂-C₁₈ alkyl sulfate) or alkyl ether sulfate (including alkyl glyceryl ether sulfates). Among the alkyl ether sulfates are those having the formula:
RO(CH₂CH₂O)ₙSO₃M

wherein R is an alkyl or alkenyl having 8 to 18 carbons, preferably 12 to 18 carbons, n has an average value of greater than 1.0, preferably greater than 3; and M is a

solubilizing cation such as sodium, potassium, ammonium or substituted ammonium. Ammonium and sodium lauryl ether sulfates are preferred.

The anionic may also be alkyl sulfosuccinates (including mono- and dialkyl, e.g., C₆-C₂₂ sulfosuccinates); alkyl and acyl taurates, alkyl and acyl sarcosinates, sulfoacetates, C₈-C₂₂ alkyl phosphates and phosphates, alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Sulfosuccinates may be monoalkyl sulfosuccinates having the formula:
R⁴O₂CCH₂CH(SO₃M)CO₂M; and

amide-MEA sulfosuccinates of the formula;
R⁴CONHCH₂CH₂O₂CCH₂CH(SO₃M)CO₂M

wherein R⁴ ranges from C₈-C₂₂ alkyl and M is a solubilizing cation.

Sarcosinates are generally indicated by the formula:
R¹CON(CH₃)CH₂CO₂M,

wherein R¹ ranges from C₈-C₂₀ alkyl and M is a solubilizing cation.

Taurates are generally identified by formula:
R²CONR³CH₂CH₂SO₃M

wherein R² ranges from C₈-C₂₀ alkyl, R³ ranges from C₁-C₄ alkyl and M is a solubilizing cation.

The inventive foaming cleansing composition may contain C₈-C₁₈ acyl isethionates. These esters are prepared by reaction between alkali metal isethionate with mixed aliphatic fatty acids having from 6 to 18 carbon atoms and an iodine value of less than 20. Preferably at least 75% of the mixed fatty acids have from 12 to 18 carbon atoms and up to 25% have from 6 to 10 carbon atoms.

The acyl isethionate may be an alkoxylated isethionate such as is described in llardi et al., U.S. Pat. No. 5,393,466, titled “Fatty Acid Esters of Polyalkoxylated isethonic acid; issued Feb. 28, 1995; hereby incorporated by reference. This compound has the general formula:
R C—O(O)—C(X)H—C(Y)H₂—(OCH—CH₂)ₘ—SO₃M⁺

wherein R is an alkyl group having 8 to 18 carbons, m is an integer from 1 to 4, X and Y are hydrogen or an alkyl group having 1 to 4 carbons and M⁺ is a monovalent cation such as, for example, sodium, potassium or ammonium.

Nonionic Surfactants

One or more nonionic surfactants may also be used in the foaming cleansing composition of the present invention.

When used, the anionic surfactant may be present at about 0.1, 0.2, 0.3, 0.5, 0.7 or 1% by wt. to about 2, 3, 4 or 5% by wt.

The nonionics which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic detergent compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxide, and the like.

The nonionic may also be a sugar amide, such as a polysaccharide amide. Specifically, the surfactant may be one of the lactobionamides described in U.S. Pat. No. 5,389,279 to Au et al. titled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

Cationic Skin Conditioning Agents

An optional component in compositions according to the invention is a cationic skin feel agent or polymer. They may be preferably used at levels of about 0.1, 0.2, 0.3% by wt. to about 0.5, 0.7, 1, 2, 3, 4, or 5% by wt.

Suitable examples include cationic celluloses. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

A particularly suitable type of cationic polysaccharide polymer that can be used is a cationic guar gum derivative, such as guar hydroxypropyltrimonium chloride (Commercially available from Rhone-Poulenc in their JAGUAR trademark series). Examples are JAGUAR C13S, which has a low degree of substitution of the cationic groups and high viscosity, JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Particularly preferred cationic polymers are JAGUAR C13S, JAGUAR C15, JAGUAR C17 and JAGUAR C16 and JAGUAR C162, especially Jaguar C13S. Other cationic skin feel agents known in the art may be used provided that they are compatible with the inventive formulation.

Cationic Surfactants

One or more cationic surfactants may also be used in the inventive foaming cleansing composition. When used they may be employed at levels of about 0.1, 0.2, 0.3, 0.5 or 1% by wt. to about 2, 3, 4 or 5% by wt.

Examples of cationic detergents are the quaternary ammonium compounds such as alkyldimethylammonium halogenides.

Other suitable surfactants which may be used are described in U.S. Pat. No. 3,723,325 to Parran Jr. titled “Detergent Compositions Containing Particle Deposition Enhancing Agents” issued Mar., 27, 1973; and “Surface Active Agents and Detergents” (Vol. I & II) by Schwartz, Perry & Berch, both of which are also incorporated into the subject application by reference.

Optional Ingredients

The compositions may further comprise antimicrobials such as 2-hydroxy-4,2′, 4′trichlorodiphenylether (DP300); preservatives such as dimethyloldimethylhydantoin (Glydant XL1000), parabens, sorbic acid etc., and the like.

Antioxidants such as, for example, butylated hydroxytoluene (BHT) and the like may be used advantageously in amounts of about 0.01% or higher if appropriate.

Microemulsions

The microemulsion composition of the invention may be further defined on the basis of various properties as follows: (1) the compositions are substantially transparent and remain stable between 5° and 45° C.; (2) the microemulsion can undergo a freeze-thaw process without affecting the emulsion, and (3) the microemulsion can dissolve at least 1% sebum, preferably at least 2% sebum, while staying in microemulsion form.

The stability of the inventive microemulsions may be and in certain respects have been characterized according to the criteria summarized in table A. Inventive compositions generally remain stable showing no major changes in pH (i.e. not greater than +/−0.4 pH units) and viscosity (not greater than +/−5,000 cps at 25 C using e.g. a rotary viscometer), no noticeable changes in transparency/translucency, and no noticeable color and odor change. Only inventive samples at Northern Light (25 C) conditions and 50° C. appeared to yellow slightly and developed a slight off-odor after 2 months.

TABLE A
Stability Test Conditions(1)

| Temperature | Time | Evaluations | Evaluation Points |
|---|---|---|---|
| Approx. 25 C. | 12 weeks to | Viscosity, | Initial |
| | 3 years | Visual | 1 day |
| | | | 1, 2, 4, 8, 12 |
| | | | weeks, 3 years. |
| 40 C. | 12 weeks | Visual only | 1, 2, 4, 8, 12 |
| | | | weeks |
| 50 C. | 1 week | Viscosity, | 1 week |
| | | Visual | |
| Approx. 25 C. | 12 weeks | Visual only | 1, 2, 4, 8, 12 |
| Northern Light | | | weeks |
| −9 C./25 C. cycle | 3 cycles | Viscosity, | 1 week |
| (24 hours at each | (6 days) | Visual on | |
| temp.) | | thawed sample | |

Note
(1)All tests performed in darkness except for Northern Light which is performed on samples stored in transparent glass jars under natural northern sky illumination through glass windows in the Northern Hemisphere..

Particle size of the inventive microemulsions of the invention are expected to be in the range of about 10 to 150 nanometers (nm) as measured via freeze fracture cryo-electron microscopy or other equivalent methods.

The compositions of the invention all form microemulsions spontaneously when diluted (mixed) with water. In many cases the diluted solutions of the invention display the Tyndall effect, i.e., reflection of blue light indicating oil droplets in the emulsion are in the range of colloidal dispersions. However, when the oil phase is relatively low in proportion to the surfactant and specific surfactant(s) is(are) used—the blue light effect may not be observed as the particle size of the oil droplets becomes smaller which further facilates a total transparency effect upon dilution.

Skin Conditioning Agents

A skin conditioning agent, also known as an emollient is defined as a substance which softens or improves the elasticity, appearance, and youthfulness of the skin (stratum corneum) by either increasing its water content, adding, or replacing lipids and other skin nutrients; or both, and keeps it soft by retarding the decrease of its water content.

The skin conditioning agent(s) or emollient(s) may consist of a single component or may be a mixture of two or more components. In addition, the emollient itself may act as a carrier for other components one may wish to add to the foaming cleansing composition.

A blend of hydrophobic and hydrophilic emollients may be used with the proviso that hydrophilic emollients are used in excess of hydrophobic emollients in the inventive foaming cleansing composition, preferably in the ratio range of 30, 20, 10 or 5 to 1 (hydrophilic to hydrophobic). Most preferably one or more hydrophilic emollients are used alone.

Hydrophilic emollients are preferably present in a concentration greater than about 0.1, 0.2, 0.3, 0.5, 0.7, or 1% by weight to a maximum of about 2, 3, 4, 5, 6, 7, 8, 9 or 10% by weight.

The term “emollient” is defined as a substance which softens or improves the elasticity, appearance, and youthfulness of the skin (stratum corneum) by either increasing its water content, adding, or replacing lipids and other skin nutrients; or both, and keeps it soft by retarding the decrease of its water content.

Useful examples of hydrophillic emollients (also known as humectants) include polyhydric alcohols, e.g. glycerine and propylene glycol, and the like; polyols such as the polyethylene glycols listed below and the like; saccharide(s) and/or polysaccharide(s) such as sucrose, sorbitol; and urea derivatives such as hydroxyethyl urea and the like may be advantageously used.

| | |
|---|---|
| Polyox WSR-205 | PEG 14M, |
| Polyox WSR-N-60K | PEG 45M, or |
| Polyox WSR-N-750 | PEG 7M. |

Preferably hydrophillic emollients will include C3 to C6 diol(s) or triol(s) or blends thereof. Advantageously these C3 to C6 diol(s) or triol(s) or blends thereof will constitute at least 20, 30, 40, 50, 60, 70, 80, or 90% by wt. of the total skin conditioning agents in the inventive composition.

Other useful examples of hydrophillic emollients include any of the following or blends thereof: alcaligenes polysaccharides; algae extract; aloe barbadensis leaf extract; bacillus/rice bran extract/soybean extract ferment filtrate; black strap powder; diglycereth-7 malate; diglycerin; diglycol guanidine succinate; erythritol; fructose; glucose; glucoronolactone; glycereth-7 glycolate; glycerin; glyceryl dimaltodextrin; glycol; hesperetin laurate; 1,2,6-hexanetriol; honey; hydrogenated honey; hydrogenated starch hydrolysate; hydrolyzed wheat protein/PEG-20 acetate copolymer; hydroxypropyltrimonium hyaluronate; inositol; lactic acid; lacitol; maltitol; maltose; mannitol; mannose; methoxy PEG-7; methoxy PEG- 10; methoxy PEG-16; methoxy PEG-25; methoxy PEG-40; methoxy PEG-100; PEG 4; PEG-6; PEG-7; PEG-8; PEG-9; PEG-10; PEG-12; PEG-14; PEG-16; PEG-18; PEG-20; PEG-32; PEG-40; PEG-45; PEG-55; PEG-60; PEG-75; PEG-90; PEG- 75; PEG-90; PEG-100; PEG-135; PEG-150; PEG-180; PEG-200; PEG-220; PEG-240; PEG-800; PEG-15 butanediol; PEG-3-methyl ether; PEG-4 methyl ether; PEG-5 pentaerythrityl ether; polyglyceryl sorbitol; potassium dextrin octenylsuccinate; potassium PCA; PPG-6 sorbeth-245; PPG-6 sorbeth-500; propylene glycol; rosa canina seed extract; sodium acetylated hyaluronate; sodium dextrin octenylsuccinate; sodium glucuronate; sodium PCA; sorbeth-6; sorbeth-20; sorbeth-30; sorbeth40; sorbitol; sorbityl silanediol; sucrose; TEA dextrin octenylsuccinate; trehalose; triglycereth-7 citrate; trioxaundecanedioic acid; tripropylene glycol; urea; urea-d-glucuronic acid; xylitol; xylose and the like.

Useful hydrophobic emollients include the following:

(a) silicone oils and modifications thereof such as linear and cyclic polydimethylsiloxanes; amino, alkyl, alkylaryl, and aryl silicone oils;

(b) fats and oils including natural fats and oils such as jojoba, soybean, sunflower, rice bran, avocado, almond, olive, sesame, persic, castor, coconut, mink oils; cacao fat; beef tallow, lard; hardened oils obtained by hydrogenating the aforementioned oils; and synthetic mono, di and triglycerides such as myristic acid glyceride and 2-ethylhexanoic acid glyceride;

(c) waxes such as carnauba, spermaceti, beeswax, lanolin, and derivatives thereof;

(d) hydrophobic plant extracts;

(e) hydrocarbons such as liquid paraffins, vaseline, microcrystalline wax, ceresin, squalene, pristan and mineral oil;

(f) higher fatty acids such as lauric, myristic, palmitic, stearic, behenic, oleic, linoleic, linolenic, lanolic, isostearic, arachidonic and poly unsaturated fatty acids (PUFA);

(g) higher alcohols such as lauryl, cetyl, stearyl, oleyl, behenyl, cholesterol and 2-hexydecanol alcohol;

(h) esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate;

(I) essential oils and extracts thereof such as mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe vera, menthol, cineole, eugenol, citral, citronelle, borneol, linalool, geraniol, evening primrose, camphor, thymol, spirantol, penene, limonene and terpenoid oils; and

(o) mixtures of any of the foregoing components, and the like.

Preferred hydrophobic emollients are selected from triglyceride oils, mineral oils, petrolatum, and mixtures thereof. Further preferred emollients are triglycerides such as sunflower seed oil.

Dispensing System and Propellants:

Suitable aerosol container dispensing systems may be preferably used to dispense the inventive compositions. These systems include lined aluminum standard aerosol cans (available from suppliers such as CCL Container (Ontario, Canada) or Exal (Buenos Aires, Argentina)) or lined 3 piece steel cans (available from suppliers such as United States Can Corp (Lombard, Ill.) or Crown Cork and Seal (Philadelphia, Pa.)).

Non-aerosol foaming dispensers known in the art or their equivalents may also be suitably used to dispense the inventive composion. A suitable dispenser may be mechanical and is generally characterized by a container for storing the composition (preferably a transparent container), a dispensing head defined by a housing containing a foam generator, optionally a pump, and usually a dip tube for transferring the composition from the container into the dispensing head. Foam may be created by requiring the composition to pass through a screen material which may be a porous substance such as a sintered material, a wire (plastic or metal) gauze screen or similar structures or other structures effective for making a foam. A suitable mechanical dispenser is a device available from the Airspray International Corporation described in PCT publication no. WO 97/13585 (Van der Heijden) incorporated herein by reference.

Other suitable foaming dispensers may include squeeze foamers. Squeeze foamer packages are well known as exemplified by the disclosures in the following patents that are incorporated herein by reference. U.S. Pat. Nos. 3,709,437, Wright, issued Jan. 9, 1973; 3,937,364, Wright, issued Feb. 10, 1976; 4,022,351, Wright, issued May 10, 1977; 4,147,306, Bennett, issued Apr. 3, 1979; 4,184,615, Wright, issued Jan. 22, 1980; 4,598,862, Rice, issued Jul. 8, 1986; and 4,615,467, Grogan et al., issued Oct. 7, 1986; and French Pat. 2,604,622, Verhulst, published Apr. 8, 1988. When squeeze foamers are used, the inventive composition is placed in the container reservoir (plastic squeeze bottle). Squeezing the container with the hand forces the composition through a foamer head, or other foam producing means, where the composition is mixed with air and then optionally through a homogenizing means that makes the foam more homogeneous and controls the consistency of the foam. The foam is then discharged as a uniform, non-pressurized aerated foam.

Dispensing systems according to the invention preferably are aerosol systems that include propellant(s). Propellant(s) are defined herein as any component or combination of components that exist as a gas at room temperature (approx. 23 C) and ambient pressure (approx. 760 torr or 101 Kpa) that is (are) capable of causing the dispensing of the inventive composition from its container. Suitable propellant(s) include preferably one or more liquifiable volatile hydrocarbons, such as isobutane or blends thereof, optionally containing dimethyl ether. The propellant is present in the packaged composition at any required and suitable level effective to dispense the inventive composition, but is preferably present in the container at levels sufficient to produce a satisfactory dispensing pressure, which will typically be 55+/−30 psig, preferably 55+/−20 psig at 23 C.

Optional Active Agents

Advantageously, active agents other than skin conditioning agents such as emollients as defined above may be added to the cleansing composition in a safe and effective amount during formulation to treat the skin during the use of the product. These active ingredients may be advantageously selected from antimicrobial and antifungal actives, vitamins, anti-acne actives; anti-wrinkle, anti-skin atrophy and skin repair actives; skin barrier repair actives; non- steroidal cosmetic soothing actives; artificial tanning agents and accelerators; skin lightening actives; sunscreen actives; sebum stimulators; sebum inhibitors; anti-oxidants; protease inhibitors; skin tightening agents; anti-itch ingredients; hair growth inhibitors; 5-alpha reductase inhibitors; desquamating enzyme enhancers; anti-glycation agents; topical anesthetics, or mixtures thereof; and the like.

These active agents may be selected from water soluble or hydrophilic active agents, oil soluble active or hydrophobic agents, pharmaceutically-acceptable salts and mixtures thereof. Advantageously the agents will be soluble or dispersible in the cleansing composition. Hydrophobic agents if present are advantageously at maximum levels of about 5, 4, 3, 2, 1, 0.5 or 0.1% by wt. The term “active agent” as used herein, means personal care actives which can be used to deliver a benefit to the skin and/or hair and which generally are not used to confer a conditioning benefit, as is conferred by humectants and emollients. previously described herein. The term “safe and effective amount” as used herein, means an amount of active agent high enough to modify the condition to be treated or to deliver the desired skin care benefit, but low enough to avoid serious side effects. The term “benefit,” as used herein, means the therapeutic, prophylactic, and/or chronic benefits associated with treating a particular condition with one or more of the active agents described herein. What is a safe and effective amount of the active agent ingredient will vary with the specific active agent, the ability of the active to penetrate through the skin, the age, health condition, and skin condition of the user, and other like factors. Preferably the composition of the present invention comprise from about 0.01% to about 50%, more preferably from about 0.05% to about 25%, even more preferably 0.1% to about 10%, and most preferably 0.1% to about 5%, by weight of the active agent component.

Anti-acne actives can be effective in treating acne vulgaris , a chronic disorder of the pilosebaceous follicles. Nonlimiting examples of useful anti-acne actives include the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, mixtures thereof and the like.

Antimicrobial and antifungal actives can be effective to prevent the proliferation and growth of bacteria and fungi. Nonlimiting examples of antimicrobial and antifungal actives include b-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, triclosan; triclocarban; and mixtures thereof and the like.

Anti-wrinkle, anti-skin atrophy and skin repair actives can be effective in replenishing or rejuvenating the epidermal layer. These actives generally provide these desirable skin care benefits by promoting or maintaining the natural process of desquamation. Nonlimiting examples of antiwrinkle and anti-skin atrophy actives include vitamins, minerals, and skin nutrients such as milk, vitamins A, E, and K; vitamin alkyl esters, including vitamin C alkyl esters; magnesium, calcium, copper, zinc and other metallic components; retinoic acid and its derivatives (e.g., cis and trans); retinal; retinol; retinyl esters such as retinyl acetate, retinyl palmitate, and retinyl propionate; vitamin B 3 compounds (such as niacinamide and nicotinic acid), alpha hydroxy acids, beta hydroxy acids, e.g. salicylic acid and derivatives thereof (such as 5-octanoyl salicylic acid, heptyloxy 4 salicylic acid, and 4-methoxy salicylic acid); mixtures thereof and the like.

Skin barrier repair actives are those skin care actives which can help repair and replenish the natural moisture barrier function of the epidermis. Nonlimiting examples of skin barrier repair actives include lipids such as cholesterol, ceramides, sucrose esters and pseudo-ceramides as described in European Pat. Specification No. 556,957; ascorbic acid; biotin; biotin esters; phospholipids, mixtures thereof, and the like.

Non-steroidal cosmetic soothing actives can be effective in preventing or treating inflammation of the skin. The soothing active enhances the skin appearance benefits of the present invention, e.g., such agents contribute to a more uniform and acceptable skin tone or color. Nonlimiting examples of cosmetic soothing agents include the following categories: propionic acid derivatives; acetic acid derivatives; fenamic acid derivatives; mixtures thereof and the like. Many of these cosmetic soothing actives are described in U.S. Pat. No. 4,985,459 to Sunshine et al., issued Jan. 15, 1991, incorporated by reference herein in its entirety.

Artificial tanning actives can help in simulating a natural suntan by increasing melanin in the skin or by producing the appearance of increased melanin in the skin. Nonlimiting examples of artificial tanning agents and accelerators include dihydroxyacetaone; tyrosine; tyrosine esters such as ethyl tyrosinate and glucose tyrosinate; mixtures thereof, and the like.

Skin lightening actives can actually decrease the amount of melanin in the skin or provide such an effect by other mechanisms. Nonlimiting examples of skin lightening actives useful herein include aloe extract, alpha-glyceryl-L-ascorbic acid, aminotyroxine, ammonium lactate, glycolic acid, hydroquinone, 4 hydroxyanisole, mixtures thereof, and the like.

Also useful herein are sunscreen actives. A wide variety of sunscreen agents are described in U.S. Pat. No. 5,087,445, to Haffey et al., issued Feb. 11, 1992; U.S. Pat. No. 5,073,372, to Turner et al., issued Dec. 17, 1991; U.S. Pat. No. 5,073,371, to Turner et al. issued Dec. 17, 1991; and Segarin, et al., at Chapter VIII, pages 189 et seq., of Cosmetics Science and Technology, all of which are incorporated herein by reference in their entirety. Nonlimiting examples of sunscreens which are useful in the compositions of the present invention are those selected from the group consisting of octyl methoxyl cinnamate (Parsol MCX) and butyl methoxy benzoylmethane (Parsol 1789), 2-ethylhexyl p-methoxycinnamate, 2-ethylhexyl N,N-dimethyl-p-aminobenzoate, p-aminobenzoic acid, 2-phenylbenzimidazole-5-sulfonic acid, oxybenzone, mixtures thereof, and the like.

Sebum stimulators can increase the production of sebum by the sebaceous glands. Nonlimiting examples of sebum stimulating actives include bryonolic acid, dehydroetiandrosterone (DHEA), orizanol, mixtures thereof, and the like.

Sebum inhibitors can decrease the production of sebum by the sebaceous glands. Nonlimiting examples of useful sebum inhibiting actives include aluminum hydroxy chloride, corticosteroids, dehydroacetic acid and its salts, dichlorophenyl imidazoldioxolan (available from Elubiol), mixtures thereof, and the like.

Also useful as actives in the present invention are protease inhibitors. Protease inhibitors can be divided into two general classes: the proteinases and the peptidases. Proteinases act on specific interior peptide bonds of proteins and peptidases act on peptide bonds adjacent to a free amino or carboxyl group on the end of a protein and thus cleave the protein from the outside. The protease inhibitors suitable for use in the present invention include, but are not limited to, proteinases such as serine proteases, metalloproteases, cysteine proteases, and aspartyl protease, and peptidases, such as carboxypepidases, dipeptidases and aminopepidases, mixtures thereof and the like.

Other useful as active ingredients in the present invention are skin tightening agents. Nonlimiting examples of skin tightening agents which are useful in the compositions of the present invention include monomers which can bind a polymer to the skin such as terpolymers of vinylpyrrolidone, (meth)acrylic acid and a hydrophobic monomer comprised of long chain alkyl (meth)acrylates, mixtures thereof, and the like.

Active ingredients in the present invention may also include anti-itch ingredients. Suitable examples of anti-itch ingredients which are useful in the compositions of the present invention include hydrocortisone, methdilizine and trimeprazineare, mixtures thereof, and the like.

Nonlimiting examples of hair growth inhibitors which are useful in the compositions of the present invention include 17 beta estradiol, anti angiogenic steroids, curcuma extract, cycloxygenase inhibitors, evening primrose oil, linoleic acid and the like. Suitable 5-alpha reductase inhibitors such as ethynylestradiol and, genistine mixtures thereof, and the like.

Nonlimiting examples of desquamating enzyme enhancers which are useful in the compositions of the present invention include alanine, aspartic acid, N methyl serine, serine, trimethyl glycine, mixtures thereof, and the like.

A nonlimiting example of an anti-glycation agent which is useful in the compositions of the present invention would be Amadorine (available from Barnet Products Distributor), and the like.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated. Physical test methods are described below:

### EXAMPLE 1

### Inventive Mousse Forming Composition

Solution A according to the invention was prepared as described in Table 1 using the procedure provided below.

TABLE 1

| | | Wt. % |
|---|---|---|
| Phase | Component | as active |
| A | DI WATER | 88.02 |
| A | Citric acid | 0.05 |
| A | Glycerin | 1.00 |
| A | DI Panthenol | 0.01 |
| A | Sodium lauroamphoacetate | 0.79 |
| A | Glydant plus | 0.15 |
| A | Polysorbate 20 | 0.50 |
| A | Butylene glycol | 0.25 |
| B | Peg-40 hydrogenated castor oil | 0.50 |
| B | Hexylene glycol | 2.00 |
| B | Benzyl alcohol | 0.40 |
| B | Bisabolol | 0.03 |
| B | Vit E acetate | 0.00 |
| B | Fragrance | 0.30 |
| C | Isobutane | 3.06 |
| C | Dimethyl ether | 0.54 |
| C | Propane | 2.40 |
| | Total | 100 |

Process Directions:
1. To the first mix tank add phase A DI water between 70-110 F (centigrade range 20-45 C)
2. Add phase A ingredients with moderate agitation
3. In a second tank add phase B ingredients starting with hexylene glycol
4. Agitate phase B until clear
5. Transfer phase B to the first tank
6. Fill solution into cans
7. Crimp valve on top of can
8. Fill propellant preblend C into can through the valve.

### EXAMPLE 2

### Inventive Mousse Foaming Compositions

Solution 1 according to the invention was prepared as described in Table 2 using the procedure provided above. Solutions 2-8 may also be prepared according to the invention as described in Table 2 according to the same procedure. In another preferred embodiment, the inventive compositions in Table 2 may be prepared without the propellant and dispensed using a mechanical foaming device or an equivalent thereof.

TABLE 2
Inventive Compositions (all amounts in wt. %)

| Component | 1 | 2 | 3 | 4 | 5 | 6 | 7 | 8 |
|---|---|---|---|---|---|---|---|---|
| DI water | To 100 | to 100 | to 100 | To 100 | to 100 | to 100 | to 100 | to 100 |
| Sodium lauroamphoacetate | 0.79 | | | | | | | |
| Cocoamidopropyl betaine - CAPB | | 1 | | | | | | |
| C12-15 Pareth-3 | | | 0.9 | | | | | |
| SLES 2 EO | | | | 0.8 | | | | |
| Disodium laureth sulfosuccinate | | | | | 0.65 | | | |
| decyl polyglucoside | | | | | | 0.8 | | |
| Linoleamidopropyl PG-Dimonium Chloride Phosphate | | | | | | | 0.75 | |
| Disodium PEG-12 dimethicone sulfosuccinate | | | | | | | | 1 |
| Polysorbate 20 | 0.5 | | | | | 0.3 | | |
| Eumulgin HPS (1) | | 0.5 | | | | | 0.4 | |
| Solubilisant LRI (2) | | | 0.5 | 0.4 | | | | 0.5 |
| PEG-40 hydrogenated castor oil | 0.5 | | | | | | | |
| Polysorbate 60 | | | | | 0.6 | | | |
| Polysorbate 80 | | | | | | 0.8 | | |
| DL-panthenol | 0.01 | | | | | | | |
| Bisabolol | 0.03 | | | | | | | |
| PEG-8 dilaurate | | 0.25 | | | 0.25 | | | 0.25 |
| Butylene glycol | 0.25 | | 0.2 | | 1.5 | 0.25 | 0.4 | |
| Hexylene glycol | 2 | | 1.6 | 2 | | | | 1.5 |
| Dipropylene glycol | | 1.5 | | 0.2 | | | | 0.3 |
| benzyl alcohol | 0.4 | 0.3 | 0.5 | | | | | |
| Glycerin | 1 | | 1 | | 0.25 | 1 | 1 | |
| Maltitol | | | | 0.85 | | | | 1 |
| Hydrogenated wheat protein | | | 0.05 | | | 0.03 | | |
| Hydroxyethyl urea | | 1 | 1 | | 0.75 | | | |
| pH adjusment (citric acid or NaOH) | (3) | (3) | (3) | (3) | (3) | (3) | (3) | (3) |
| Presenvative (parabens, hydantoins, etc) | 0.1-0.5 | 0.1-0.5 | 0 1-0.5 | 0.1-0.5 | 0.1-0.5 | 0.1-0.5 | 0.1-0.5 | 0.1-0.5 |
| Fragrance, color | 0.1-1 | 0.1-1 | 0.1-1 | 0.1-1 | 0.1-1 | 0.1-1 | 0.1-1 | 0.1-1 |
| Propellant blend (4) | 0-15 | 0-15 | 0-15 | 0-15 | 0-15 | 0-15 | 0-15 | 0-15 |

Notes
(1) Blend of Coceth-7, PPG-1-PEG-9 Lauryl Glycol Ether and PEG-40 Hydrogenated Castor Oil.
(2) Blend of PPG-26-Butheth-26 & PEG-40 Hydrogenated Castor Oil.
(3) As needed to attain a pH in the range of 6 to 7.
(4) Propellant blend may include one or more of the following: Hydrocarbons such as Isobutane, n-pentane, isopentane, hexane and mixtures thereof, and nonhydrocarbons such as carbon dioxide, nitrous oxide, dimethyl ether, fluorochlorohydrocarbons, and mixtures thereof and the like

### EXAMPLE 3

### Comparative Mousse Foaming Compositions

Solutions 1A to 3A may be as prepared as described in Table 3 using the procedure provided above. It is expected that these samples will be disadvantageous for the following reasons: 1A: will provide too much lather and therefore will be difficult to wipe off without rinsing with water; 2A: will likely irritate the skin to a greater extent than the inventive compositions due to the amount of anionic surfactant present as can be demonstrated by various art recognized techniques such as described in U.S. Ser. No. 67/664,991 and U.S. Pat. No. 6,664,217 hereby incorporated by reference in their entirety; 3A: will likely leave the skin tacky due to the substantial amount of skin conditioning agents and/or cationic polymers that are present.

TABLE 3
Comparative Compositions (all amounts in wt. %)

| Component | 1A | 2A | 3A |
|---|---|---|---|
| DI water | to 100 | to 100 | to 100 |
| sodium lauroamphoacetate | 5 | | |
| Cocoamidopropyl betaine - CAPB | | | |
| C12-15 Pareth-3 | | | 0.9 |
| SLES 2 EO | | .65 | |
| disodium laureth sulfosuccinate | | | |
| decyl polyglucoside | | | |
| Linoleamidopropyl PG-Dimonium Chloride | | | |
| Phosphate | | | |
| disodium PEG-12 dimethicone | | | |
| sulfosuccinate | | | |
| polysorbate 20 | 0.5 | | |
| Eumulgin HPS (1) | | 0.5 | |
| Solubilisant LRI (2) | | | 0.5 |
| PEG-40 hydrogenated castor oil | 0.5 | | .5 |
| polysorbate 60 | | | .5 |
| polysorbate 80 | | | .5 |
| DL-panthenol | 0.01 | | |
| bisabolol | 0.03 | | |
| PEG-8 dilaurate | | 0.25 | |
| butylene glycol | 0.25 | | 1 |
| hexylene glycol | 2 | | 1.6 |
| dipropylene glycol | | 1.5 | |
| benzyl alcohol | 0.4 | 0.3 | 0.5 |
| glycerin | 1 | 3 | 10 |
| maltitol | | | |
| hydrogenated wheat protein | | | 0.05 |
| hydroxyethyl urea | | 1 | 1 |
| pH adjusment (citric acid or NaOH) | as | as | as |
| | needed | needed | needed |
| preservative (e.g. parabens, hydantoins, | 0.1-0.5 | 0.1-0.5 | 0.1-0.5 |
| etc) | | | |
| fragrance, color | 0.1-1 | 0.1-1 | 0.1-1 |
| Propellant blend (4) | 0-15 | 0-15 | 0-15 |

Notes:
see Table 2

Test Methods:
Foam Density Determination Method

Foam density may be measured using a stainless steel pycnometer. First, the pycnometer is rinsed with tap water followed by distilled water. Than it is rinsed with acetone, dried, and allowed to come to room temperature (approx. 23 C). The empty pycnometer is tare weighed. To calculate the volume of the pycnometer, the body of the pycnometer is carefully filled with water, avoiding air bubbles. The cover is placed on and collar screwed into place. Excess water is wiped away and the filled pycnometer is weighed.

After cleaning and drying, the product for which the density is to be measured is added to the pycnometer and the cover is carefully pressed down until seated. Excess sample is expelled through the center is wiped away and the collar is screwed on. Excess sample is cleaned from the outside of the pycnometer and the filled pycnometer is weighed. Density is calculated using the following equation:
ρ=g sample/g H₂O.

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

What is claimed is:

1. A mild foaming cleansing composition comprising:
a. amphoteric surfactant(s) in a total concentration range of about 0.1 to 5% by wt.;
b. anionic surfactant(s) in a total concentration that is less than the amphoteric surfactant(s) and greater than about 0.01% by wt.;
c. propellant(s) in a total concentration range of about 0 to about 15% by wt.
d. less than 5% by wt. of a hydrophobic skin conditioning agent; and
e. hydrophilic emollients in the concentration range of about 0.1 to 10% by wt.

2. (canceled)

3. The composition of claim 1 wherein the hydrophilic emollients include at least 1% by wt. of C3 to C6 diol(s) or triol(s) or blends thereof.

4. The composition of claim 1 wherein the propellants are selected from hydrocarbons, dimethyl ether, carbon dioxide, nitrous oxide, compressed air, nitrogen, fluorohydrocarbons, or blends thereof.

5. The composition of claim 1 wherein the composition is a microemulsion.

6. The composition of claim 1 wherein the total surfactant concentration is less than about 5% by wt.

7. The composition of claim 1 wherein the nonionic surfactant concentration is in the range of about 0.1 to about 5% by wt.

8. The composition of claim 1 wherein the density of the foam dispensed by a dispenser measured 5 seconds after dispensing is at least about 0.1 g/ml (at approx. 23 C and approx. 760 torr or 101 Kpa).

9. The composition of claim 1 wherein the soap content is less than about 5% by wt.

10. The composition of claim 1 further comprising one or more skin active agent(s) in the total concentration range of about 0.02 to 3% by wt.

11. The composition of claim 10 wherein the one or more skin active agent(s) are selected from antibacterial agent(s), anti-aging agent(s), anti-acne agent(s) or skin firming agent(s).

12. A mild mousse foaming packaged cleansing product comprising:
a. a container having a valve,
b. a low surfactant cleansing composition contained in the container including:
1. amphoteric surfactant(s) in a total concentration range of about 0.1 to 5% by wt.;
2. anionic surfactant(s) in a total concentration that is less than the amphoteric surfactant(s) and greater than about 0.01% by wt;
3. propellant(s) in a total concentration range of about 0.5 to about 10% by wt.,
4. less than 5% by wt. of a hydrophobic skin conditioning agent,
5. hydrophilic emollients in the concentration range of about 0.1 to 10% by wt; and
c. wherein the valve is capable of releasing the cleansing composition when the internal pressure exerted by the cleansing composition is greater than the outside air pressure.

13. A mild foaming packaged cleansing product comprising:
a. A container having a non-aerosol mechanical dispenser,
b. a low surfactant cleansing composition contained in the container including:
1. amphoteric surfactant(s) in a total concentration range of about 0.1 to 5% by wt.;
2. anionic surfactant(s) in a total concentration that is less than the amphoteric surfactant(s) and greater than about 0.01% by wt;
3. less than 5% by wt. of a hydrophobic skin conditioning agent,
4. hydrophilic emollients in the concentration range of about 0.1 to 10% by wt; and
c. wherein the dispenser is capable of dispensing the cleansing composition as a foam.

14. A method for cleansing the skin without rinsing with water, comprising the steps of:
a. applying the mild foaming cleansing composition of claim 1 to the skin;
b. distributing the applied composition to the area of skin desired; and
c. wiping off the applied composition.
